(19) United States
(12) Patent Application Publication
(10) Pub. No.: US 2004/0086477 A1
(43) Pub. Date: May 6, 2004

(54) Method of and kit for mitigating cradle cap

(76) Inventors: Wanda Richard, Ada, MI (US); Rebecca Whipple, Grant, MI (US); Richard Jentis, Bridgewater, NJ (US)

Correspondence Address: THOMAS HOXIE, NOVARTIS, CORPORATE INTELLECTUAL PROPERTY, ONE HEALTH PLAZA 430/2, EAST HANOVER, NJ, 07936-1080, US

(21) Appl. No.: 10/667,214

(22) Filed: Sep. 19, 2003

Related U.S. Application Data
(60) Provisional application No. 60/412,877, filed on Sep. 23, 2002.

(51) Int. Cl. A61K 7/06; A61K 7/11; A61K 31/01

(52) U.S. Cl. 424/70.11; 514/762

(57) ABSTRACT

Methods and kits for removing scales caused by cradle cap on the heads of infants are disclosed and include topically applying to the scalp an effective amount of a pre-treatment composition, a shampoo and a moisturizer for respective time periods sufficient to remove cradle cap. A scale-removing device is also used to help remove and loosen any scales.

This application claims the benefit of U.S. Provisional Application No. 60/412,877, filed Sep. 23, 2002.

### FIELD OF THE INVENTION

The present invention relates to methods and kits for the removal of scales on the scalp of an infant caused by cradle cap. More particularly, the methods and kits relate to the use of a four step method that facilitates the removal of such scales.

1. Background of the Invention

Cradle cap, or infantile seborrhoeic dermatitis, is a common condition of the scalps of babies. Cradle cap manifests itself as greasy and oily plaques or scales on the scalp. The onset of cradle cap usually beings in the first few weeks of life and disappears over a period of weeks or months. Although the condition is benign, many parents prefer to have cradle cap eliminated, or at a minimum mitigated, because of the unsightly appearance of the plaques and scales. Currently various treatments are recommended for treating cradle cap. For example, some pediatricians recommend using a mineral oil to loosen the scales and plaque followed by gentle combing with a fine-toothed cup. Other pediatricians recommend using shampoos that contain actives, such as zinc, ketaconazole or salicylic acid.

Although a parent can separately obtain the individual components from a store, parents are forced to buy larger quantities of material than is necessary to remove the cradle cap from the infant's scalp. Moreover, the parent needs to spend additional time to seek out each individual component. Finally, once the components are individually purchased, the parent needs to consult an additional source, for example a pediatrician or book, to find out how best to use the various components to treat the cradle cap.

With all of the changes in a parent's life with the birth of a new child, parents need to be provided with a fast and quick method of procuring the necessary products along with literature to treat the cradle cap. Thus, it is an object of the present invention to provide a kit that is consumer friendly and convenient to facilitate the treatment and removal of cradle cap.

### SUMMARY OF THE INVENTION

The present invention features a kit that can be used by consumer to mitigate the effects of cradle cap. Specifically, the cradle cap kit includes at least four components, a pre-treatment composition, a scale-removing device, a shampoo and a moisturizer, all packaged into a single secondary package. The pre-treatment composition, for example a gelled mineral oil, is applied to the scalp of an infant to loosen the scales caused by cradle cap. A scale-removing device, e.g., a comb, is subsequently used to remove the loosened scales. A shampoo is the used to wash away the pre-treatment composition and the scales and to ultimately cleanse the infant's scalp. Finally, a moisturizer is used on the infant's scalp to restore the infant's scalp to its natural suppleness and pliability.

2. Detailed Description of the Invention

The present invention features a method and kit for treating cradle cap in the scalps of infants. What is meant by “treating” and “treatment” is lessening the appearance or effects of the plaques or scales on the scalp, e.g., physical removal. The kit includes at least four components a pre-treatment composition, a scale removing device, a shampoo for gently cleaning the scalp of the infant and a moisturizer, all of which are packaged together in a single secondary package.

The first component of the kit of the present invention is a topical pre-treatment composition. The pre-treatment composition, e.g., contains an oil material or emollient, that can easily dissolve or loosen the plaques or scales or a formulation thereof. Oil materials, refer to compounds that have low solubility in water, e.g., less than about 1% by weight at 25° C. The percentage of the oil materials in the pre-treatment composition can range from about 10% to about 100% by weight based on the weight of the pre-treatment composition. Although the pre-treatment composition can contain water, e.g., in an emulsion, it is preferable that the pre-treatment composition be of a single phase, oil.

Examples of an oil material/emollient includes, but is not limited to, mineral oil, petrolatum, C₇-C₄₀branched chain hydrocarbons, C₁-C₃₀alcohol esters of C₁-C₃₀carboxylic acids, C₁-C₃₀alcohol esters of C₂-C₃₀dicarboxylic acids, monoglycerides of C₁-C₃₀carboxylic acids, diglycerides of C₁-C₃₀carboxylic acids, triglycerides of C₁-C₃₀carboxylic acids, ethylene glycol monoesters of C₁-C₃₀carboxylic acids, ethylene glycol diesters of C₁-C₃₀carboxylic acids, propylene glycol monoesters of C₁-C₃₀carboxylic acids, propylene glycol diesters of C₁-C₃₀carboxylic acids, C₁-C₃₀carboxylic acid monoesters and polyesters of sugars, polydialkylsiloxanes, polydiarylsiloxanes, polyalkarylsiloxanes, cyclomethicones having 3-9 silicon atoms, vegetable oils, hydrogenated vegetable oils, polypropylene glycols, polypropylene glycol C₄-C₂₀alkyl ethers, di-C₈-C₃₀alkyl ethers, and mixtures thereof.

Examples of straight- and branched-chain hydrocarbons having from about 7 carbon atoms to about 40 carbon atoms include, but are not limited to, dodecane, isododecane, squalane, cholesterol, hydrogenated polyisobutylene, docosane, hexadecane and isohexadecane.

Examples of C₁-C₃₀alcohol esters of C₁-C₃₀carboxylic acids and of C₂-C₃₀dicarboxylic acids include, but are not limited to, straight- and branched-chain materials, as well as aromatic derivatives. Also suitable are esters such as monoglycerides of C₁-C₃₀carboxylic acids, diglycerides of C₁-C₃₀carboxylic acids, triglycerides of C₁-C₃₀carboxylic acids, ethylene glycol monoesters of C₁-C₃₀carboxylic acids, ethylene glycol diesters of C₁-C₃₀carboxylic acids, propylene glycol monoesters of C₁-C₃₀carboxylic acids and propylene glycol diesters of C₁-C₃₀carboxylic acids. Straight-chain, branched-chain and aryl carboxylic acids are included herein. Also suitable are propoxylated and ethoxylated derivatives of these materials. Non-limiting examples include diisopropyl sebacate, diisopropyl adipate, isopropyl myristate, isopropyl palmitate, myristyl propionate, ethylene glycol distearate, 2-ethylhexyl palmitate, isodecyl neopentanoate, C₁₂-C₁₅alcohols benzoate, di-2-ethylhexyl maleate, cetyl palmitate, myristyl myristate, stearyl stearate, cetyl stearate, behenyl behenrate, dioctyl maleate, dioctyl sebacate, diisopropyl adipate, cetyl octanoate, diisopropyl dilinoleate, caprylic/capric triglyceride, PEG-6 caprylic/capric triglyceride, PEG-8 caprylic/capric triglyceride and mixtures thereof.

Examples of silicones include, but are not limited to, polydialkylsiloxanes, polydiarylsiloxanes, polyalkarylsiloxanes and cyclomethicones having 3-9 silicon atoms are useful oils. Either volatile or nonvolatiles silicones are suitable in the pre-treatment composition.

Examples of vegetable oils and hydrogenated vegetable oils include, but are not limited to, castor oil, cotton seed oil, pine oil, sunflower oil, corn oil, sesame oil, palm kernel oil, canola oil, coconut oil, safflower oil, peanut oil and any hydrogenated derivatives thereof and mixtures thereof.

A preferred oil material for use in the pre-treatment composition is mineral oil which is a mixture of liquid hydrocarbons obtained from petroleum. Since the pre-treatment composition is massaged into the scalp in order to moisturize and loosen the cradle cap scales, there is a risk of the mineral oil dripping from the scalp and into the eyes, nose and/or mouth of the infant. To minimize this from happening, it is preferable for the mineral oil to be gelled. Gelled mineral oils are blends of a hydrocarbon mineral oil with di-block co-polymers and/or tri-block co-polymers. For example, the components of the blend in a gelled mineral oil are described in U.S. Pat. No. 5,221,534 to DesLauriers et al., which is hereby incorporated by reference in its entirety. A preferred gelled mineral oil for use in the present invention is mineral oil blended with ethylene/propylene/styrene copolymer and butylene/ethylene/styrene co-polymer. The gelled mineral oil can be present in the pre-treatment composition at a concentration from about 50% to about 99%. Such a gelled mineral oil is sold by Penreco (Houston, Tex.) under the trademark VERSAGEL.

The pre-treatment composition should have a viscosity such that it does not easily drip into the eyes, nose and/or mouth of the infant yet fluid enough to allow the pre-treatment composition to flow from its packaging. For example, the pre-treatment composition should have a viscosity from about 3,450 to about 3,600 cps measured using a RVT viscometer with spindle TA at 10 rpm.

The second component in the cradle cap kit of the present invention is a scale-removing device. Such a device includes, but is not limited to, brushes and combs. As used herein a brush is a handled device that has bristles used for smoothing, sweeping or orienting hair. As used herein a comb is a device that has teeth used for adjusting, confining or cleaning hair. Preferably, the scale removing device is a comb that has teeth that are in a density of about sixteen teeth per inch. The comb, e.g., is manufactured by injection molding such that there is no flashing or sharp edges or points remaining on the comb.

The third component of the present invention is a topical shampoo. For example, the shampoo is a composition that contains a detersive surfactant, or a surfactant that is able to provide a cleansing or detergent benefit. The detersive surfactant is preferably comprised of, based upon the total weight of the shampoo, from about 5% to about 20%, e.g., from about 10% to about 15%. The detersive surfactant, e.g., includes, but is not limited to, nonionic surfactants, anionic surfactants, cationic surfactants, amphoteric surfactants and mixtures thereof. The component, e.g., be a mild baby shampoo that is commercially-available. The term “mild” as used herein refers to the amount of irritation of the eyes and/or skin of an infant caused by the application or use of the baby shampoo. Examples of commercially available baby shampoos suitable for use as the third component of the present invention include, but are not limited to, GERBER Baby Shampoo available from Gerber Products Company (Fremont, Mich.), JOHNSON'S BABY Shampoo available from Johnson & Johnson Consumer Companies, Inc. (Skillman, N.J.) and BABY MAGIC Baby Shampoo available from Playtex Products, Inc. (Westport, Conn.).

Examples of non-ionic surfactants suitable for use in the shampoo include, but are not limited to, long-chain alkyl glucosides or polyglucosides, which are the condensation products of a C₈-C₃₀long-chain alcohol with a sugar or starch polymer; alkylene oxide esters of fatty acids which are the condensation products of alkylene oxides with fatty acids; the condensation products of alkylene oxides with fatty acids; alkylene oxide diesters of fatty acids which are the condensation products of alkylene oxides with two moles of fatty acids; and alkylene oxide ethers of fatty alcohols which are the condensation products of alkylene oxides with fatty alcohols.

The term “amphoteric”, as used herein, means either molecules that contain both acidic and basic sites or zwitterionic molecules which possess both positive and negative charges within the same molecule. Examples of zwitterionic compounds include, but are not limited to, alkyl betaines and amidoalkyl betaines. Examples of amphoteric surfactants include, but are not limited to, amphocarboxylates, alkyl betaines, amidoalkyl betaines, amidoalkyl sultaines, amphophsophates, phosphobetaines, pyrophosphobetaines, carboxyalkyl alkyl polyamines and mixtures thereof.

Examples of anionic surfactants include, but are not limited to, alkyl sulfates, alkyl ether suifates, alkyl monoglyceryl ether sulfates, alkyl monoglyceride sulfates, alkyl monoglyceride sulfonates, alkyl sulfonates, alkylaryl sulfonates, alkyl sulfosuccinates, alkyl ether sulfosuccinates, alkyl sulfosuccinamates, alkyl amidosulfosuccinates, alkyl carboyxlates, alkyl amidoethercarboxylates, alkyl succinates, fatty acyl sarcosinates, fatty acyl amino acids, fatty acyl taurates, fatty alkyl sulfoacetates and alkyl phosphates.

Examples of cationic surfactants include, but are not limited to, cationic ammonium salts and amino-amides.

In addition to the detersive surfactant, the balance of the shampoo composition can include one or more of the following ingredients water, a pearlescent, an opacifying agent, a thickener, a conditioner, a humectant, a chelator, a dye, a fragrance and a pH adjuster.

The shampoo is formulated to be “mild” which means that the shampoo does not irritate the eyes, nose or mouth of an infant. Any skilled artisan in the toiletry arts can formulate the shampoo to meet these mildness requirements.

The fourth component of the present invention is a topical moisturizer. The topical moisturizer, e.g., is a composition that contains moisturizing agents in a suitable base. The purpose of the moisturizer is to help reduce the appearance or signs of flakiness on the infant's scalp due to cradle cap. Furthermore, the moisturizer helps to keep the scalp moisturized by retaining water. The moisturizer remains in/on the hair and/or scalp after application and is not rinsed away.

Examples of suitable bases include, but are not limited to, creams, ointments, oils, waxes, gels, sticks, balms, lotions, suspensions, dispersion and emulsions. The emulsions can be two phase, such as oil-in-water or water-in-oil, or three phase emulsions, such as oil-in-water-in-oil and water-in-oil-in-water. The aggregate concentration of the moisturizing agents in the moisturizer should range from about 0.1% to about 80% by weight of the moisturizer, e.g., from about 5% to about 70%, e.g., from about 10% to about 50%. The moisturizer is non-toxic and non-irritating to an infant's scalp, nose, eyes and/or mouth.

Moisturizing agents include at least one emollient and/or humectant. An emollient is a compound that helps to main the soft, smooth and pliable appearance of the skin. The emollient can, e.g., remain on the stratum corneum of the epidermis and acts as a lubricant. Examples of emollients include, but are not limited to, hydrocarbons, fatty acids, fatty alcohols and esters. Additionally, any of the aforementioned oil materials disclosed in connection with the pre-treatment composition can also serve as an emollient in the moisturizer.

A humectant is a compound that increases the water content of the epidermis. Examples of humectants include, but are not limited to, guanidine; alpha-hydroxy acids and the salts thereof; beta-hydroxy acids and the salts thereof; polyhydroxy acids and the salts thereof; aloe vera; polyhydroxy alcohols, such as sorbitol, glycerol, hexanetriol, propylene glycol, butylene glycol and hexylene glycol; polyethylene glycols; sugars and starches and derivatives thereof; hyaluronic acid; and mixtures thereof.

Preferably, the moisturizer is a light yet substantive moisturizer that can be easily massaged into the scalp of an infant. The moisturizer, for example, contains butters which are heavy emollients. The term “heavy emollients” as used herein refers to emollients that are solid at room temperature (approximately 25° C.); this is in contrast to “oil” which as used herein refers to emollients that are liquid at room temperature. Examples of butters include, but are not limited to, shea butter, cocoa butter, mango butter, illipe butter, jojoba butter and avocado butter. Such butters provide a higher viscosity then moisturizers with oils alone. For example, the moisturizer should have a viscosity from about 9,000 to about 14,000 cps measured using a RVT viscometer with spindle TA at 10 rpm.

Each of the chemical components of the present invention, i.e., the pre-treatment composition, the shampoo and the moisturizer is individually packaged in a primary packaging. Any suitable primary packaging can be used, e.g., bottles, tubes, pumps, aerosols and canisters. Primary packaging refers to the containers that are in physical contact with the contents therein.

In addition to the four primary components of the present invention: the pre-treatment composition, the scale-removing device, the shampoo and the moisturizer, a booklet or pamphlet describing or providing directions on how to use the components is provided. For example, the pamphlet can set forth the following steps:

a) loosen the cradle cap scales the pre-treatment composition;

b) remove cradle cap scales with scale removing device;

c) cleanse with shampoo; and

d) moisturize the scalp with a moisturizer.

The four primary components with the optional pamphlet are packaged together into a secondary package. Preferred secondary packages include cartons, cylindrical tubes, and boxes. The secondary package should be easily handled by a consumer and convenient for retailers to display on their store shelves. For example, the secondary packages should be of geometrical dimensions, e.g., having rectangular, square or circular tops and bottoms, that allow them to be easily displayed or stored on a store shelf, e.g., stacked or placed flush side-by-side. The secondary package contains the various primary packages.

### EXAMPLE

Set forth below are examples of each of pre-treatment composition, shampoo and moisturizer respectively.

Pre-Treatment Composition

| Ingredient | Weight Percent (%) |
|---|---|
| Part A | |
| Mineral oil | q.s. |
| Mineral oil (and) | 28 |
| ethylene/propylene/styrene | |
| copolymer (and) | |
| butylene/ethylene/styrene copolymer | |
| Part B | |
| Isodecyl neopentanoate | 0.5 |
| Liquid petrolatum | 0.2 |
| Sweet almond oil | 0.1 |
| Safflower oil | 0.1 |
| Preservative | 0.1 |
| | 100 |

The pre-treatment composition is made by mixing the ingredients under Part A at 70° C. until uniform. The ingredients under Part B are heated to 60° C. until the mixture is clear and uniform. The ingredients of Part B are then mixed to those of Part A at 70° C. Once the combination of Parts A and B are uniform, the mixture is cooled to 45° C.

Shampoo

| | Weight |
|---|---|
| Ingredient | Percent (%) |
| Part A | |
| Water | q.s. |
| Acrylate/C₁₀₋₃₀ alkyl (acrylate cross-polymer) | 0.5 |
| Sodium hydroxide (10% solution) | 1 |
| Part B | |
| Sodium laureth sulfate | 15 |
| PEG-2 stearate | 1.5 |
| PEG-150 distearate | 0.5 |
| Preservative | 0.5 |
| Decyl glucoside | 2 |
| Water | 13.5 |
| Styrene/PVP co-polymer | 1 |
| Cocamidopropyl betaine | 7 |
| | 100 |

In Part A, disperse the carbomer into the water and let stand. Neutralize with the sodium hydroxide solution and heat the mixture to 50° C. Heat the ingredients in Part B to 60° C. Phase Part B into Part A. Cool while stirring.

Moisturizer

| Ingredient | Weight Percent (%) |
|---|---|
| Part A | |
| Water | q.s. |
| Hydroxyethyl cellulose | 0.8 |
| Sodium stearyl pthalamate | 0.8 |
| Citric acid | 0.05 |
| Part B | |
| Caprylic/capric triglyercides | 8 |
| Isodecyl neopentanoate | 5 |
| Glycerol stearate | 3.5 |
| Cocoa butter | 0.1 |
| Shea butter | 0.1 |
| Mango butter | 0.5 |
| Petrolatum | 0.5 |
| Carnauba wax | 0.5 |
| Sorbitan monoleate | 5 |
| | 100 |

Heat Part B to 80° C. while gently mixing until all of the waxes and butters are melted and uniform. Heat Part A to 75° C. Phase Part B into Part A while stirring and continue mixing with unforced cooling. Continue stirring until mixture cools to room temperature or about 25° C.

Each of the components are individual packaged into primary packages, e.g., bottles. The pre-treatment composition, shampoo and moisturizer along with the scale removing device are assembled together into a carton to form the cradle cap kit of the present invention. An instruction booklet detailing use of the individual components can also be enclosed within the carton.

A consumer purchases the cradle cap kit of the present invention and uses the kit to alleviate the cradle cap on the infant's scalp. To use the kit, the consumer opens the carton and removes the contents. First, the pre-treatment composition is applied to the affected area of the infant's scalp. The pre-treatment composition is gently massaged into the scalp such that it moisturizes and looses the cradle cap skills. The consumer takes care in avoiding contact the eyes, nose and mouth of the infant with the pre-treatment composition. Next, the consumer gently uses the scale-removing device, e.g., a comb, to further loosen and remove the scales. The scale-removing device should be cleansed with warm water and cleanser, e.g., the shampoo, after use. Third, the shampoo is thoroughly massaged into the baby's scalp to clean the scalp and remove the pre-treatment composition. The scalp of the infant is rinsed with clear water. If necessary, the scalp-removing device should be used again to remove any remaining scales. Last, a small, pea-sized amount (approximately 0.5 mL) of the moisturizer is gently massaged into the scalp of the infant. Once again, the consumer should take care to not contact the eyes, nose and mouth of the infant with the moisturizer.

It is understood that while the present invention has been described in conjunction with the detailed description thereof that the foregoing description is intended to illustrate and not limit the scope of the invention, which is defined by the scope of the following claims. Other aspects, advantages and modifications are within the scope of the claims.

What is claimed is:

1. A method for mitigating the presence of scales on an infant's scalp caused by cradle cap comprising the sequential steps of:
a) loosening said scales by massaging a pre-treatment composition into said scalp of an infant;
b) using a scale-removing device to remove said scales from said scalp;
c) cleaning said scalp by using a shampoo; and
d) applying a moisturizer to said scalp,
wherein said pre-treatment composition, said scale-removing device, said shampoo and said moisturizer are included in a single secondary package.

2. The method of claim 1, wherein said pre-treatment composition comprises an oil material.

3. The method of claim 2, wherein said oil material is mineral oil.

4. The method of claim 3, wherein said oil material is gelled mineral oil.

5. The method of claim 1, wherein said scale-removing device is a comb.

6. The method of claim 1, wherein said moisturizer further comprises a butter.

7. The method of claim 1, wherein said moisturizer has a viscosity from about 9,000 to about 14,000 cps as measured by a RVT viscometer with a TA spindle and rotating at 10 rpm.

8. A kit for mitigating the effects of cradle cap on the scalp of an infant comprising:
a) a secondary package containing at least four components that comprise a pre-treatment composition;
b) a scale-removing device;
c) a shampoo; and
d) a moisturizer,
wherein said pre-treatment composition, said shampoo and said moisturizer are each individually packaged in a primary package.

9. The kit of claim 8, further comprising instructions to perform the method of claim 1.

10. The kit of claim 8, wherein said pre-treatment composition comprises an oil material.

11. The kit of claim 8, wherein said oil material is a mineral oil.

12. The kit of claim 11, wherein said oil material is a gelled mineral oil.

13. The kit of claim 8, wherein said scale-removing device is a comb.

14. The kit of claim 8, wherein said moisturizer further comprises a butter.

15. The kit of claim 8, wherein said moisturizer has a viscosity from about 9,000 to about 14,000 cps as measured by a RVT viscometer with a TA spindle and rotating at 10 rpm.

16. The kit of claim 8, wherein said primary packaging is a carton.
